(19) United States
(12) Patent Application Publication
(10) Pub. No.: US 2024/0304312 A1
(43) Pub. Date: Sep. 12, 2024

(54) METHOD OF DOCUMENTING EXPOSURE TO INFECTED PATIENTS

(71) Applicants: Manuel Granado, Rio Rancho, NM (US); Tamara Granado, Rio Rancho, NM (US)

(72) Inventors: Manuel Granado, Rio Rancho, NM (US); Tamara Granado, Rio Rancho, NM (US)

(21) Appl. No.: 18/117,833

(22) Filed: Mar. 6, 2023

(51) Int. Cl. G16H 40/20; G06K 19/077; G16H 50/80; H04W 4/80

(52) U.S. Cl. CPC G16H 40/20; G06K 19/07758; G16H 50/80; H04W 4/80

(57) ABSTRACT

A method of tracking a location of a person in a medical facility for tracking the location of a person in a medical facility includes positioning an identification badge on the person and tracking the location of the identification badge throughout the medical facility using RFID readers that detect an RFID tag in the identification badge when it draws near. The method includes steps for documenting time a healthcare team member spends with a patient and tracking exposure to infectious diseases.

### CROSS-REFERENCE TO RELATED APPLICATIONS

Not Applicable

### STATEMENT REGARDING FEDERALLY SPONSORED RESEARCH OR DEVELOPMENT

Not Applicable

### THE NAMES OF THE PARTIES TO A JOINT RESEARCH AGREEMENT

Not Applicable

### INCORPORATION-BY-REFERENCE OF MATERIAL SUBMITTED ON A COMPACT DISC OR AS A TEXT FILE VIA THE OFFICE ELECTRONIC FILING SYSTEM

Not Applicable

### STATEMENT REGARDING PRIOR DISCLOSURES BY THE INVENTOR OR JOINT INVENTOR

Not Applicable

### BACKGROUND OF THE INVENTION

### (1) Field of the Invention

The disclosure relates to tracking methods and more particularly pertains to a new tracking method for tracking the location of a person in a medical facility.

### (2) Description of Related Art Including Information Disclosed Under 37 CFR 1.97 and 1.98

The prior art relates to security systems that use radio frequency identification to grant access to and monitor the location of people within a facility. The prior art also relates to devices which document time that a caregiver spends with a patient and security systems which record time and attendance of individuals.

### BRIEF SUMMARY OF THE INVENTION

An embodiment of the disclosure meets the needs presented above by generally comprising positioning an identification badge on a person. The identification badge includes a radio frequency identification (RFID) tag having an identification code associated with the identification badge. The identification badge is positioned within a threshold range of a selected RFID reader of a plurality of RFID readers. An identification signal comprising the identification code is transmitted from the identification badge to the selected RFID reader. At least one location signal comprising the identification code and an indication of proximity to the selected RFID reader is transmitted from the selected RFID reader to a remote recording system. A location of the identification badge and a time period during which the identification badge is positioned in the location are determined based on the at least one location signal. The location and the time period are recorded on the remote recording system.

There has thus been outlined, rather broadly, the more important features of the disclosure in order that the detailed description thereof that follows may be better understood, and in order that the present contribution to the art may be better appreciated. There are additional features of the disclosure that will be described hereinafter and which will form the subject matter of the claims appended hereto.

The objects of the disclosure, along with the various features of novelty which characterize the disclosure, are pointed out with particularity in the claims annexed to and forming a part of this disclosure.

### BRIEF DESCRIPTION OF SEVERAL VIEWS OF THE DRAWING(S)

The disclosure will be better understood and objects other than those set forth above will become apparent when consideration is given to the following detailed description thereof. Such description makes reference to the annexed drawings wherein:

FIG. 1 is a perspective view of an identification badge and a radio frequency identification reader according to an embodiment of the disclosure.

FIG. 2 is a rear view of an identification badge according to an embodiment of the disclosure.

FIG. 3 is a block diagram of an identification badge and a radio frequency identification reader according to an embodiment of the disclosure.

FIG. 4 is an in-use view of an identification badge according to an embodiment of the disclosure.

FIG. 5 is an in-use view of an identification badge and a radio frequency identification reader according to an embodiment of the disclosure.

### DETAILED DESCRIPTION OF THE INVENTION

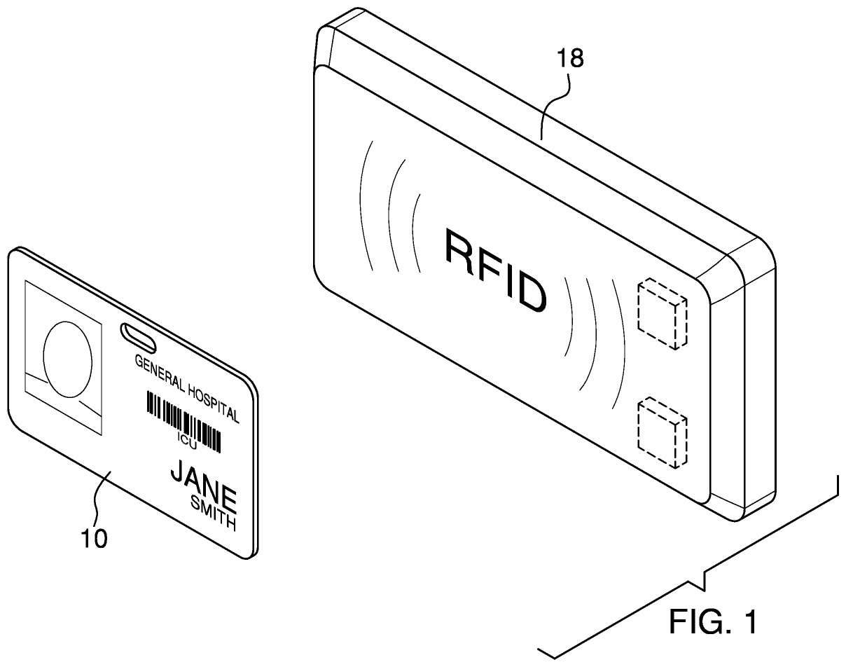

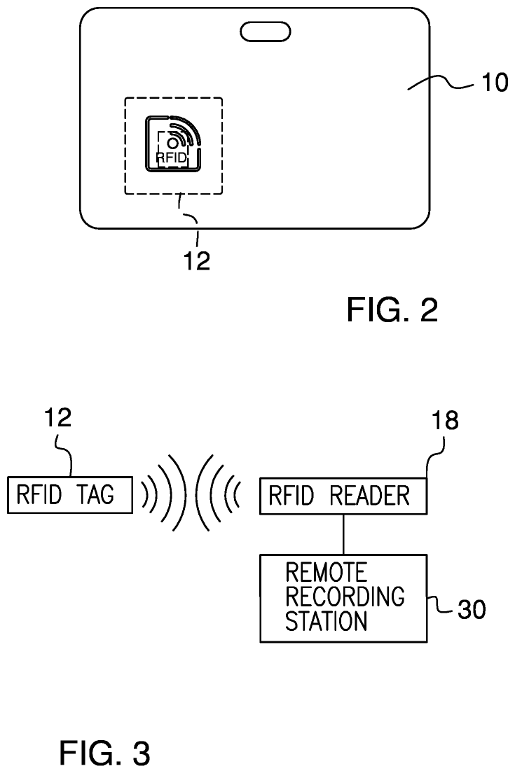

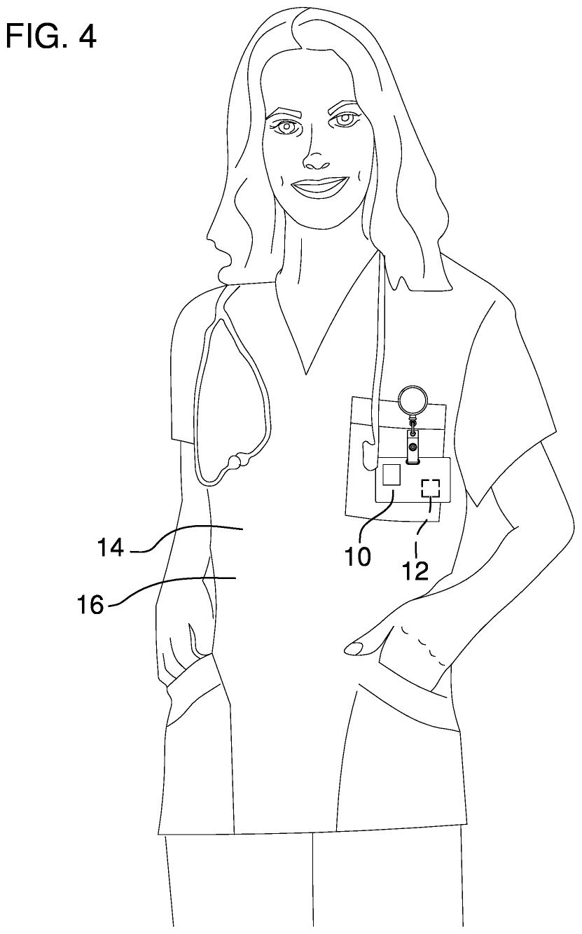

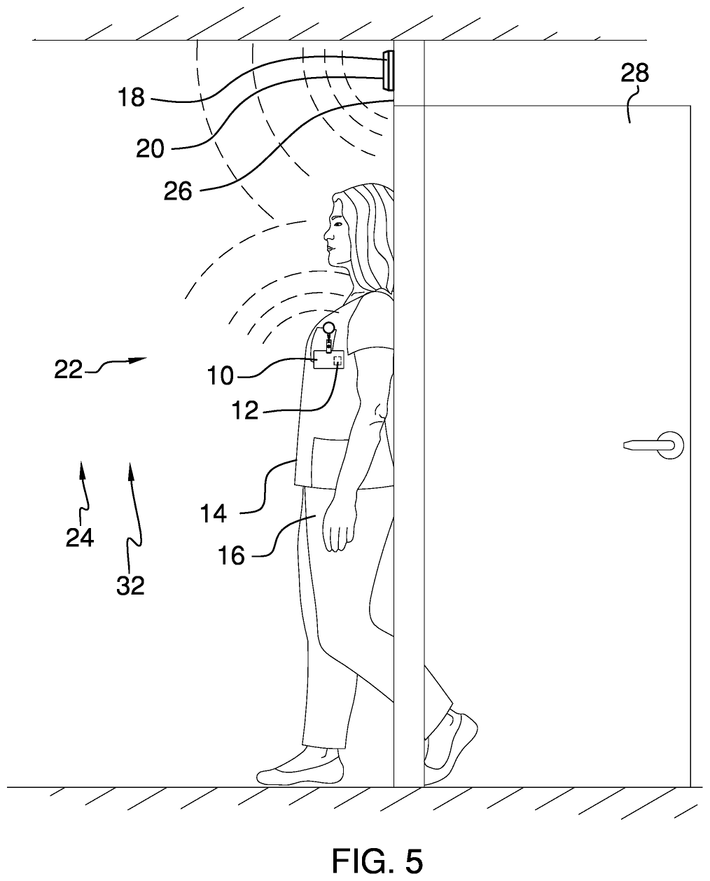

With reference now to the drawings, and in particular to FIGS. 1 through 5 thereof, a new tracking method embodying the principles and concepts of an embodiment of the disclosure will be described.

As best illustrated in FIGS. 1 through 5, the method of tracking a location of a person in a medical facility generally comprises positioning an identification badge 10 with a radio frequency identification (RFID) tag 12 on the person 14. The RFID tag 12 has an identification code associated with the identification badge 10. The person 14 may be a healthcare team member 16 such as a physician, nurse, patient care technician, dietician, housekeeper, or the like. The identification code may also be an employee identification number associated with the person 14. Positioning the identification badge 10 within a threshold range of a selected RFID reader 20 of a plurality of RFID readers 18. The “threshold range” is a zone where the RFID reader 18 is capable of receiving a transmission from the RFID tag 12. The threshold range of each RFID reader 18 may be positioned adjacent to one of a plurality of patient care areas 22. In the specification and the claims, a “patient care area” is a zone which is intended for a patient to occupy during medical treatment, and may include a room 24, a hospital bed, a designated chair, an operating table, or the like. Also, each RFID reader 18 may be mounted on one of a plurality of surfaces 26 and adjacent to one of a plurality of doors 28. A tracking system in which each RFID reader 18 is positioned in this way might be designed to detect when the identification badge 10 passes through one of the plurality of doors 28, in which case a computer program of the system could determine the location of the identification badge 10 based on which RFID reader 18 detected the identification badge 10 most recently and the order in which multiple RFID readers 18 detected the identification badge 10.

The method further comprises transmitting an identification signal comprising the identification code from the identification badge 10 to the selected RFID reader 20. Then at least one location signal comprising the identification code of the identification badge 10 and an indication of proximity to the selected RFID reader 20 is transmitted from the selected RFID reader 20 to a remote recording system 30. A location 32 of the identification badge 10 and a time period during which the identification badge 10 is positioned in the location 32 is determined based on the at least one location signal. “Time period” as used in the specification and in the claims may include one or more of a span of time spent in the location 32, an entry time when the identification badge 10 is first detected in the location 32 and an exit time when the identification badge 10 is last detected in the location 32. The location 32 and the time period are recorded on the remote recording system 30. Additionally, the location 32 and the time period may be recorded in an electronic medical record of a medical patient associated with the location 32.

In some embodiments, the method may also comprise steps directed to track exposure to infectious diseases. In such embodiments, the method includes designating an area containing an individual with an infectious disease as an infectious area on the remote recording system 30. When the location 32 of the identification badge 10 coincides with the infectious area, an exposure event is recorded which includes the location 32 of the identification badge 10 and an exposure period during which the exposure event occurred. The method may also include informing the person 14 of the exposure event.

With respect to the above description then, it is to be realized that the optimum dimensional relationships for the parts of an embodiment enabled by the disclosure, to include variations in size, materials, shape, form, function and manner of operation, assembly and use, are deemed readily apparent and obvious to one skilled in the art, and all equivalent relationships to those illustrated in the drawings and described in the specification are intended to be encompassed by an embodiment of the disclosure.

Therefore, the foregoing is considered as illustrative only of the principles of the disclosure. Further, since numerous modifications and changes will readily occur to those skilled in the art, it is not desired to limit the disclosure to the exact construction and operation shown and described, and accordingly, all suitable modifications and equivalents may be resorted to, falling within the scope of the disclosure. In this patent document, the word “comprising” is used in its non-limiting sense to mean that items following the word are included, but items not specifically mentioned are not excluded. A reference to an element by the indefinite article “a” does not exclude the possibility that more than one of the element is present, unless the context clearly requires that there be only one of the elements.

I claim:

1. A method of tracking a location of a person in a medical facility, the method comprising:
positioning an identification badge on the person, the identification badge including a radio frequency identification (RFID) tag having an identification code associated with the identification badge;
positioning the identification badge within a threshold range of a selected RFID reader of a plurality of RFID readers;
transmitting an identification signal comprising the identification code from the identification badge to the selected RFID reader;
transmitting at least one location signal comprising the identification code and an indication of proximity to the selected RFID reader from the selected RFID reader to a remote recording system;
determining a location of the identification badge and a time period during which the identification badge is positioned in the location from the at least one location signal; and
recording the location and the time period on the remote recording system.

2. The method of claim 1, wherein the person is a healthcare team member.

3. The method of claim 2, further comprising recording the location and the time period in an electronic medical record of a medical patient associated with the location.

4. The method of claim 1, wherein the threshold range of each RFID reader is positioned adjacent to one of a plurality of patient care areas.

5. The method of claim 1, wherein each RFID reader is mounted on one of a plurality of surfaces and adjacent to one of a plurality of doors.

6. The method of claim 1, further comprising:
designating an area containing an individual with an infectious disease as an infectious area on the remote recording system; and
recording an exposure event when the location of the identification badge coincides with the infectious area, wherein the exposure event includes the location of the identification badge and an exposure period during which the exposure event occurred.

7. The method of claim 6, further comprising determining the exposure event occurred previously to the step of designating the area as an infectious area on the remote recording system.

8. The method of claim 6, further comprising informing the person of the exposure event.

9. A method of tracking a location of a person in a medical facility, the method comprising:
positioning an identification badge on the person, the identification badge including a radio frequency identification (RFID) tag having an identification code associated with the identification badge, wherein the person is a healthcare team member;
positioning the identification badge within a threshold range of a selected RFID reader of a plurality of RFID readers, wherein each RFID reader is mounted on one of a plurality of surfaces and adjacent to one of a plurality of doors;
transmitting an identification signal comprising the identification code from the identification badge to the selected RFID reader;
transmitting at least one location signal comprising the identification code and an indication of proximity to the selected RFID reader from the selected RFID reader to a remote recording system;
determining a location of the identification badge and a time period during which the identification badge is positioned in the location from the at least one location signal;
recording the location and the time period on the remote recording system; and
recording the location and the time period in an electronic medical record of a medical patient associated with the location.

10. The method of claim 9, further comprising:
designating an area containing an individual with an infectious disease as an infectious area on the remote recording system; and
recording an exposure event when the location of the identification badge coincides with the infectious area, wherein the exposure event includes the location of the identification badge and an exposure period during which the exposure event occurred.

11. The method of claim 10, further comprising:
determining the exposure event occurred previously to the step of designating the area as an infectious area on the remote recording system; and
informing the person of the exposure event.
